(19) United States
(12) Patent Application Publication
(10) Pub. No.: US 2009/0304771 A1
(43) Pub. Date: Dec. 10, 2009

(54) Local Delivery System for the Chemotherapeutic Drug Paclitaxel

(75) Inventors: Anthony M. Lowman, Wallingford, PA (US); Vanessa Vardon, Philadelphia, PA (US); James Schuster, Narberth, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Drexel University, Philadelphia, PA (US)

(21) Appl. No.: 12/417,160

(22) Filed: Apr. 2, 2009

Related U.S. Application Data
(60) Provisional application No. 61/072,830, filed on Apr. 3, 2008.

(51) Int. Cl. A61F 2/00; A61K 31/337; A61P 35/00

(52) U.S. Cl. 424/423; 514/449

(57) ABSTRACT

The present invention provides methods for producing a semi-degradable polymeric composite drug delivery device for localized delivery of chemotherapeutic agents to be used in conjunction with total vertebral body replacement surgery that requires placement of a vertebral replacement cage for the treatment of a spinal neoplasm.

### CROSS-REFERENCE TO RELATED APPLICATIONS

This application claims priority under 35 U.S.C. §119(e) to U.S. Provisional Application No. 61/072,830, filed on Apr. 3, 2008, which application is incorporated by reference herein in its entirety.

### BACKGROUND OF THE INVENTION

Primary cancer tumors often metastasize to other parts of the body where they can form secondary tumors at a site distant to that of the original cancer. Among the most common sites for metastases, the skeletal system is third, with up to 40% of all metastases presenting themselves in the bone (Singh et al., 2006, J. Bone Joint Surg. Br. 88-B:434-442; Vrionis et al., 2003, Neurosurg. Focus 15:E12; Heary et al., 2001, Neurosurg. Focus 11:E1). Approximately 60-70% of skeletal metastases occur in the vertebral column. Up to 10% of the 1.2 million cancer patients diagnosed annually will suffer from spinal cord compression resulting from a secondary skeletal tumor (Vrionis et al., 2003, Neurosurg. Focus 15:E12; Jacobs et al., 2001, Neurosurg Focus 11:E10). As the survival rates of cancer patients continue to increase, the number of spinal metastases will also increase (Jacobs et al., 2001, Neurosurg Focus 11:E10).

The most common primary cancers that spread to the skeletal system are lung, breast, prostate, renal, and thyroid (Singh et al., 2006, J. Bone Joint Surg. Br. 88-B:434-442; Heary et al., 2001, Neurosurg. Focus 11:E1). Breast cancer is the most common type of cancer among women, with an estimated 182,460 newly diagnosed cases and 40,480 deaths in the United States for 2008 (National Cancer Institute). This type of cancer also has the highest incidence of spinal metasases, accounting for 39.3% of secondary spinal neoplasms (Singh et al., 2006, J. Bone Joint Surg. Br. 88-B:434-442). It has been shown that 69% of all patients dying of breast cancer also have bone metastases and that the mortality rate increases to 70% following the development of bone metastases (Cicek and Oursler, 2006, Cancer Metastasis Rev. 25:635-644; Guise, 2000, Cancer 88:2892-2898). The prostate is the most common site for neoplasm diagnoses in men, and is second only to lung cancer as the leading cause of death among men in the United States. Up to 90% of patients that die from prostate cancer are found to have skeletal metastases during autopsy (Keller, et al., 2001, Cancer Metastasis Rev. 20:333-349). These numbers are significant because they show the prevalence of spinal tumors in a large population of cancer patients whose quality of life is an important consideration.

Bone metastases in the vertebral bodies often lead to debilitating pain due to osteolytic processes. There is evidence of both osteoblastic (bone forming) and osteoclastic (bone consuming) damage in different types of skeletal metastases (Cicek and Oursler, 2006; Keller, et al., 2001, Cancer Metastasis Rev. 20:333-349; Cancer Metastasis Rev. 25:635-644; Guise, 2000, Cancer 88:2892-2898). This damage can lead to subsequent fractures, nerve impingement, and in extreme cases, partial or total paralysis due to spinal cord compression.

Patients in the advanced stages of metastatic cancer traditionally have not received aggressive treatment, with mostly palliative measures carried out in an effort to improve quality of life. These measures include any combination of chemotherapy, radiotherapy, resection or removal of the tumor, and stabilization of the spine.

Radiation therapy involves the use of high energy radiation to kill cancer cells and shrink tumors. It can either be completed using a machine outside of the body as the source or by placing radioactive substances into the body near the target cells. Cells can also be targeted and pretreated with a radiosensitizing agent prior to external radiotherapy to enhance the effects of the procedure. Radiotherapy can only be used to treat pain and is ineffective in treating spinal instability. For this reason it remains an adjuvant treatment shown to be most effective in combination with surgery. Radiation therapy can actually lead to further bone compromise in some cases (Heary et al., 2001, Neurosurg. Focus 11:E1).

Surgery is now considered a viable option for most patients with a life expectancy greater than 12 weeks (Heary et al., 2001, Neurosurg. Focus 11:E1; Hussein et al., 2001, Eur. J. Surg. Oncol. 27:196-199). It has been found successful for stopping and even reversing progressive neurological deficits, providing pain relief, increasing spinal stability, and in turn preventing subsequent deformities and pathological fracture (Liu et al., 2003, Neurosurg. Focus 15:E2. Surgical treatments can range from total vertebral body replacements to minimally invasive procedures such as percutaneous vertebroplasty where bone cement is injected into the anterior portion of the damaged vertebrae in an effort to provide stabilization. Due to the already compromised nature of the vertebral bone however, it is not surprising that the incidence of cement leakage following vertebroplasty has been found to be as high as 72.2% (Singh et al., 2006, J. Bone Joint Surg. Br. 88-B:434-442).

Most common surgical methods include some combination of the following: radical resection of the tumor, insertion of a prosthetic vertebral body, bone grafting, and anterior or posterior stabilization (or both; Patchell et al., 2005, Lancet 366:643-648; Ma et al., 1987, Clin. Orthop. Relat. Res. 215:78-90; Ernstberger et al., 2005, Arch. Orthop. Trauma Surg. 125:660-669; Ernstberger et al., 2005, ACTA Orthop. Belg. 71:459-466). Removal of the tumor and stabilization usually leads to significant improvements in the patients' pain with success rates anywhere from 89-100% for moderate pain relief (Jacobs et al., 2001, Neurosurg. Focus 11:E10; Yao et al., 2003, Neurosurg. Focus 15:E6).

The basic procedure involves the either total or partial removal of the damaged vertebral body and tumor followed by the placement of a prosthetic titanium vertebral body cage and adjunct supporting systems such as titanium rods and pedicle screws. However, due to the invasive nature of surgical intervention, there is a one month post-operative period before oncological treatment can commence to address both the primary tumor and any remnant cancer cells in the area surrounding the excised neoplasm. This period following surgical treatment, when oncological treatment must be suspended, is a critical time which could ideally be utilized for further tumor suppression.

Clearly there is an urgent need for new treatments of metastatic and primary cancers of the spine. The present invention fills this need.

### SUMMARY OF THE INVENTION

In one embodiment, the present invention comprises a sustained release local delivery system comprising a hydrogel exo-structure having at least one biocompatible polymer, wherein the polymer further comprises at least one pharmacological agent. In one aspect, the polymer is bioabsorbale. In another aspect, the polymer comprises a fibrous mat. In still another aspect, the mat comprises a pharmaceutical agent different from that of other respective mats. In another aspect, the mat is made from an electrospinning process. In yet another aspect, the polymer comprises polyvinyl alcohol, poly(lactide), poly(lactide-co-glycolide), or a combination thereof. In still another aspect, pharmaceutical agent comprises Paclitaxel. In another aspect, the pharmaceutical agent is released at a rate of at least 8.5% per day.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph depicting released myoglobulin (%) from 50/50 PLGA electrospun fibers as a function of time (days) over the course of 61 days.

### DETAILED DESCRIPTION OF THE INVENTION

The present invention is related to a composite drug delivery device comprising a permanent vertebral replacement cage and a semi-degradable polymeric composite drug delivery device. The drug delivery device allows the local delivery of at least one therapeutic agent useful in the treatment of spinal cancer. The therapeutic agent of the instant invention comprises the chemotherapy agent Paclitaxel.

The present invention further comprises methods of treating a cancer of the spine by removing a cancerous vertebrae and replacing it with a composite drug delivery device comprising a permanent vertebral replacement cage and a semi-degradable polymeric composite drug delivery device for the local delivery of Paclitaxel.

### Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

The term “anticancer drug” as used herein is defined as a drug for the treatment of cancer, such as for a solid tumor. The anticancer drug preferably reduces the size of the tumor, inhibits or prevents growth or metastases of the tumor, and/or eliminates the tumor.

The term “brachytherapy” as used herein is defined as insertion of a radioactive source into a patient in the form of tiny pellets, or seeds, which are implanted directly into a tumor-containing organ.

The term “cross-linking agent” as used herein is defined as an entity which creates chemical bonds, called cross links, between two separate molecules. In a specific embodiment, the cross-linking agent is a salt of a divalent cation. In a preferred embodiment, the cross-linking agent is calcium chloride. A cross-linking composition is a composition containing a cross-linking agent.

The term “drug” as used herein is defined as a compound which aids in the treatment of disease or medical condition or which controls or improves any physiological or pathological condition associated with the disease or medical condition. In a specific embodiment, the drug is an anticancer drug.

The term “hydrogel” as used herein is defined as a composition generated in situ in a body from a water-soluble biodegradable and biocompatible polymer and a cross linking agent.

The term “in situ” as used herein is defined as restricted to a specific site within a body without substantial invasion of surrounding tissues.

The term “local treatment” as used herein is defined as providing therapy to a specific and defined area of a body. In a preferred embodiment, the therapy is restricted primarily to this area and does not extend to nearby areas or tissues. In another preferred embodiment, the region is a solid tumor.

The term “therapeutic agent” as used herein is defined as an agent which provides treatment for a disease or medical condition. The agent in a specific embodiment improves at least one symptom or parameter of the disease or medical condition. For instance, in tumor therapy, the therapeutic agent reduces the size of the tumor, inhibits or prevents growth or metastases of the tumor, or eliminates the tumor. Examples include a drug, such as an anticancer drug, a gene therapy composition, a radionuclide, a hormone, a nutriceutical, or a combination thereof.

The term “tumor” as used herein is defined as an uncontrolled and progressive growth of cells in a tissue. A skilled artisan is aware other synonymous terms exist, such as neoplasm or malignancy. In a specific embodiment, the tumor is a solid tumor. In other specific embodiments, the tumor derives, either primarily or as a metastatic form, from cancers such as of the liver, prostate, pancreas, head and neck, breast, brain, colon, adenoid, oral, skin, lung, testes, ovaries, cervix, endometrium, bladder, stomach, and epithelium (such as a wart) and metastasizes to the spine.

### Description:

The present invention provides methods for producing a semi-degradable polymeric composite drug delivery device for localized delivery of chemotherapeutic agents to be used in conjunction with, but not limited to, total vertebral body replacement surgery that requires placement of a vertebral replacement cage.

In one embodiment, the composite drug delivery device releases at least one chemotherapy agent to the immediate surrounding area of an excised vertebral neoplasm.

In another embodiment, composite drug delivery device releases Paclitaxel to the immediately surrounding area of an excised vertebral neoplasm.

In still another embodiment, composite drug delivery device releases Paclitaxel in conjunction with at least one other therapeutic agent useful in the treatment of spinal neoplasm.

The skilled artisan will readily appreciate that local delivery of Paclitaxel, either alone or in combination with another therapeutic agent, such as anti-inflammatory agents (steroidal and non-steroidal) or pain management agents including, but not limited to, acetaminophen, aspirin, ibuprofen, and opiates (including morphine, hydrocodone, codeine, fentanyl, methadone), would be useful in treating spinal neoplasm.

### I. Compositions

The present invention comprises a composite drug delivery device comprising at least two components: a permanent component and a drug-loaded delivery component. The permanent component comprises a vertebral replacement cage that protects the spinal cord, preserves vertebral spacing, and stabilizes the spinal column. The drug delivery component comprises a biocompatible polymer that had been loaded with a therapeutic agent. In one embodiment the drug is Paclitaxel. The polymer may be degradable, non-degradable (or permanent), or semi-permanent.

### A. Degradable Drug Delivery Component

All polymers degrade, but the time scale of degradation as compared to that of the application they are suited for varies. Those polymers which are considered degradable have erosion times on a smaller or similar order to their application lifespan (Gopferich 1996). There are three main types of degradable polymers. Type 1 are water soluble polymers which possess degradable crosslinks which impart insolubility until dissolvation in an aqueous environment. Type 2 polymers are water insoluble but possess pendent side groups which are responsible for solubilization following hydrolysis, ionization, or protonation in an aqueous environment. Type 3 are water insoluble polymers which possess hydrolytically unstable linkages in their backbone. These polymers are cleaved into smaller oligomers and monomers in an aqueous environment (Laurencin, 1997). The well known polyesters such as poly(lactic acid), PLA, and poly(lactic-co-glycolic acid), PLGA, are examples of Type 1 degradable polymers along with polyvinylpyrrolidine, PVP. Polyanhydrides possess an unstable anhydride linkage in their background and thus are Type 3 polymers along with poly(e-caprolactone) and poly(amino acids) (Ibim et al., 1997, Biomaterials 18:1565-1569; Leong et al., 1985, J. Biomed. Mater. Res. 19:941-955; Attawia et al., 1995, J. Biomed. Mater. Res. 29:1233-1240).

Degradable polymers are further separated according to their erosion characteristics into surface or heterogeneously eroding and bulk eroding (Gopferich, 1996, Biomaterials 17:103-114; Gopferich, 1997, Biomaterials 18:397-403; von Burkersroda et al., 1997, Biomaterials 18:1599-1607; von Burkersroda et al., 2002, Biomaterials 23:4221-4231). Degradation is the process by which polymer chains are cleaved into oligomers and eventually monomers and is characterized by the decrease in polymer chain molecular weight. Erosion however is the process through which polymers experience mass loss due to the evacuation of oligomers and monomers. The distinction between surface and bulk eroding polymers is made based upon whether the polymer exhibits uniform constant surface erosion or rather by nonrconstant spontaneous erosion from the inner core. This behavior is determined by the hydrolysis half life of the compositional functional groups and backbone linkages (Gopferich, 1996, Biomaterials 17:103-114).

Polymers that possess functional groups with relatively short hydrolysis half lives tend to be surface eroding because degradation via hydrolysis is extremely rapid relative to water diffusion into the polymer core thus degradation is concentrated at the interface between the polymer and aqueous environment. Polyanhydrides and poly(ortho esters) are examples of surface eroding polymers (Gopferich, 1996, Biomaterials 17:103-114; Gopferich, 1997, Biomaterials 18:397-403; von Burkersroda et al., 1997, Biomaterials 18:1599-1607; von Burkersroda et al., 2002, Biomaterials 23:4221-4231).

Bulk eroding polymers exhibit relatively slow hydrolysis therefore allowing for water diffusion into the polymer core and degradation throughout the polymer before erosion results. These polymers tend to maintain mass until spontaneously eroding. Two phenomena are thought to contribute to this spontaneous erosion. Autocatalysis is the process by which the acidic degradation products accumulate in the inner core of the polymer and thus contribute to the further degradation of the center in relation to the surface. Percolation phenomena results from the inability of degradation products to leave the core of the polymer matrix until a critical degree of erosion has occurred creating pores allowing for their release.

Implants made from biodegradable and biocompatible polymers such as poly(lactide), PLA, and poly(lactide-co-glycolide), PLGA, have been studied extensively as vehicles for the delivery of therapeutic agents. These polymers are ideal for processing into drug delivery vehicles because they degrade via hydrolytic cleavage of the ester bonds in the polymer chains. The acid monomers and oligomers formed by the polymer degradation enhance further breakdown. Thus the degradation rate of these polymers can be altered by changing multiple variables such as the molecular weight of the polymer chains (longer chains allow for more bond breakages before loss of structural integrity), the polymer content of the implants (glycolide is more hydrophilic than lactide thus higher glycolide content makes for faster degradation), and the implant density of (less dense allows for faster diffusion of water into the core facilitating degradation but also allows for better diffusion of degradation products out effectively decreasing degradation rate) (Panyam et al., 2003, J. Control. Release 92:1-2).

### B. Non-Degradable Drug Delivery Component

Drug delivery scaffolds may be permanent, or non-degradable. One common type of polymer scaffold is called a hydrogel. Hydrogels are 3-dimensional, water swollen polymer networks that are made insoluble by cross-links. These crosslinks can be either chemical or physical in nature and determine the polymer structural characteristics. These networks can be composed of up to 90% water and are ideal for drug delivery applications due to their swelling behavior. Some important parameters that can affect drug release from hydrogels are the network mesh size or pore size as well the interconnectivity of the pores and the hydrodynamic radius of the diffusing solute (Lowman and Peppas, 1999, J. Biomat. Sci. POlym. Ed. 10:999-1099; Peppas et al., 1999, J. Control. Release 62:81-87). In the present invention, hydrogels may comprise mats of fibers of varying orientations, including parallel orientations, radial orientations, or any other fiber orientation that provides the optimal drug-loading and drug release profile.

There are two main processing methods for the fabrication of PVA hydrogels. One method requires the use of a chemical solvent such as glyocal, gluteraldehyde, or borate to chemically crosslink linear polymer chains (Lowman and Peppas, 1999, J. Biomat. Sci. POlym. Ed. 10:999-1099; Peppas et al., 1999, J. Control. Release 62:81-87; Ogomi et al., 2005, J. Control. Release 103:315-323). As with any material intended for biomedical uses, it is desirable to minimize the amount of residual solvents present in the implant. An alternative solvent-free method for creating crosslinked PVA hydrogels is through the use of a freeze-thawing technique. This technique creates crystalline areas within the PVA which act as sites for semi-permanent physical crosslinks. There are a variety of parameters that can be altered to affect the structural characteristics of the PVA hydrogels via the freeze-thawing technique. These include the molecular weight of the PVA, the polymer solution concentration, the number of freeze-thaw cycles, the temperature extremes the polymer is exposed to, and the length of time of both the freezing and thawing steps. It has been shown that increasing the number of freeze-thaw cycles results in increased rigidity and strength of the hydrogel due to the increase in regions of crystallinity (Mongia et al., 1996, J. Biomat. Sci. Polym. Ed. 7:1055-1064; Lozinsky et al., 2001, Bioseparation 10:163-188; Lozinsky et al., 2003, Trends Biotechnol. 21:445-451).

Covalently cross-linked poly(vinyl alcohol) (PVA) gels can be produced by making a physically associated PVA hydrogel that has a crystalline phase, forming covalent crosslinks by exposing the physically associated PVA hydrogel to an effective amount of ionizing radiation, and removing the physical associations by exposure to a temperature above the melting point of the physically associated crystalline phase to produce a covalently cross-linked vinyl polymer hydrogel. The physical properties of the produced hydrogel can be adjusted by varying controlled parameters such as the proportion of physical associations, the concentration of polymer and the amount of radiation applied. PVA covalently cross-linked vinyl polymer hydrogels can be made translucent, preferably transparent, or opaque depending on the processing conditions. The stability of the physical properties of the produced hydrogel can be enhanced by controlling the amount of covalent crosslinks.

Such PVA hydrogels can be made to have a wide range of mechanical properties, such as very low to moderately high compressive moduli. Critical to the final modulus is the number of physical associations present in the precursor gels. A large number of physical associations serves to reduce the total yield of the radiation induced crosslinks, reducing the final modulus of the material. Thus, weakly associated precursor physical gels produce stronger covalently cross-linked vinyl polymer hydrogels. This phenomenon allows control of the final material properties by modulation of the physical associations in the precursor gel.

The porosity and pore size in covalently cross-linked vinyl polymer hydrogels can be controlled in that the melt-out step removes physical associations, leaving voids of controllable volume. This is not possible by direct irradiation of PVA solutions. In addition, upon completion of the processing, they will be inherently sterile due to the irradiation processing.

Polyvinyl alcohols are commonly divided into “fully hydrolyzed” and “partly hydrolyzed” types, depending on how many mole-percent of residual acetate groups remain in the molecule. Polyvinyl alcohols can be manufactured from polyvinyl acetate by alcoholysis using a continuous process. By varying the degree of polymerization of the polyvinyl acetate and its degree of hydrolysis (saponification) a number of different grades can be supplied. Typically, suitable polyvinyl alcohols for the practice of the present invention have a degree of hydrolysis (saponification) of about 80-100 percent, preferably about 95-99.8 percent. The degree of polymerization of suitable polyvinyl alcohols for the practice of the present invention is in the range of about 100 to about 50,000, preferably about 1,000 to about 20,000.

Crosslinks in PVA gels may be either covalent (chemical) crosslinks or physical associations (physical). Covalent crosslinks are formed typically through chemical modification, or through irradiation. Physical associations may be formed via freeze-thaw cycling, dehydration or through controlled manipulation of the solubility of the vinyl polymer in a solvent (to produce a “thetagel”), disclosed in U.S. published patent application Ser. No. US20040092653 or by a combination of such methods. In general, the formation of a thetagel includes a step of mixing the vinyl polymer solution with a gellant, wherein the resulting mixture has a higher Flory interaction parameter than the vinyl polymer solution. In the present invention, both covalent and physical associations can be employed, in that a physically cross-linked precursor gel will be covalently crosslinked by irradiation.

The use of irradiation to form covalent crosslinks has several advantages over chemical crosslinking. Chemical crosslinking is often performed by the addition of a reactive metallic salt or aldehyde and subjecting the system to thermal radiation. For example, crosslinking may be performed by adding (di-)isocyanates, urea-/phenolic-melamine-resins, epoxies, or (poly-)aldehydes. However, the use of such reagents for chemical crosslinking can leave residues that decrease the biocompatibility of the PVA hydrogel.

Crosslink formation by irradiation of polymers in solution is a suitable method for the generation of hydrogels for biomedical use. Crosslinking via an ionization source provides adequate control of the reaction, a lower number of unwanted processes (e.g. homografting of monomer to the side of a polymer chain) and generates an end product suitable for use with little additional processing or purification. The irradiation and sterilization steps can often be combined.

Permeability, porosity, and interconnectivity are all important characteristics of a PVA hydrogel because they influence the release rate of incorporated molecules.

### C. Semi-Permanent Drug Delivery System

A semi-permanent drug delivery system can be created by combining a degradable component with a more permanent scaffold. One such multi-component system can be created by embedding degradable drug-loaded microparticles into a permanent PVA hydrogel.

The hydrogels used for the purpose of this research do not need to meet any specific mechanical guidelines due to their placement in a titanium vertebral cage which will be loadbearing. They also do not need a specified porosity for tissue in-growth, thus allowing for enhanced flexibility in processing techniques and parameters in order to achieve the desired microparticle loading and distribution.

Another possibility for controlling release from permanent PVA hydrogels is through the use of biodegradable coatings on the exterior of the gel. Such coatings can help to prevent initial drug release known as the burst effect by acting as barriers to drug diffusion. Adding an additional coating on the surface of the implant may prove to be easier than adjusting the processing parameters of the degradable insert or the PVA hydrogel itself. Also, adjusting the processing parameters of the constituents while possibly decreasing the burst effect will probably not succeed in halting it all together. Some possible coatings to be explored include a high polymer concentration PLGA solution as well as calcium carbonate.

The drug release profile for the instant invention includes a release rate of at least 1-50% per day, including all integers encompassed therein. In one embodiment, the drug is released at a rate of at least 1-20% per day, including all whole or partial integers encompassed there between. In still another embodiment, the drug is released at a rate of at least 1-10% per day. In one embodiment the drug is released at a rate of at least 8.5% per day. The present invention contemplates the use of different drug release devices that may be loaded with different therapeutic agents and have differing release profiles for each therapeutic agent.

### D. Pharmacological Agents and Compositions: Paclitaxel

The invention encompasses cremophor-free formulations comprising Paclitaxel, derivatives, or pharmaceutically acceptable salts thereof, as well as solubilizers. Paclitaxel solubilizers of the invention include any compound that facilitates solubilzation of Paclitaxel in an aqueous medium, and include the classes of PEG-Vitamin Es; quaternary ammonium salts; PEG-monoacid fatty esters; PEG-glyceryl fatty esters; polysorbates; PEG-fatty alcohols. These formulations are advantageous in that they do not contain cremophor and thus avoid or reduce the toxicities and other disadvantages of cremophor formulations. The formulations of the invention also solubilize Paclitaxel in aqueous medium and thus are particularly advantageous because Paclitaxel is practically insoluble in water.

The formulations of the invention are useful for treating mammalian cancers and other medical conditions treatable by Paclitaxel. By “treating” it is meant that the formulations are administered to inhibit or reduce the rate of cancer-cell proliferation in an effort to induce partial or total remission, for example, inhibiting cell division by promoting microtubule formation. Examples of cancers treatable or preventable by formulations of the invention include, but are not limited to, cancers of the spine. The mode, dosage, and schedule of administration of Paclitaxel, derivatives, and pharmaceutically acceptable salts thereof in human cancer patients have been extensively studied, see, e.g. 1989 Ann. Int. Med., 111:273-279.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Formulations of a pharmaceutical composition suitable for use in the present invention comprise the active ingredient combined with a pharmaceutically acceptable carrier, such as sterile water or sterile isotonic saline. Formulations for administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile aqueous or oily suspension or solution. This suspension or solution may be formulated according to the known art, and may comprise, in addition to the active ingredient, additional ingredients such as dispersing agents, wetting agents, or suspending agents described herein. Such sterile formulations may be prepared using a non-toxic biocompatable diluent or solvent, such as water or 1,3-butane diol, for example. Other acceptable diluents and solvents include, but are not limited to, Ringer's solution, isotonic sodium chloride solution, and fixed oils such as synthetic mono- or diglycerides. Other biocompatable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### II. Methods

### A. Electrospinning

Polymer scaffolds may be generated by any method known in the art. In one embodiment, a polymer scaffold is generated by electrospinning (Kim et al., 2004, J. Control. Release 98:47-56; Kim et al., 2003, Biomaterials 24:4977-4985; Xu et al., 2005, J. Control. Release 108:33-42; Zeng et al., 2003, J. Control. Release 92:227-231; Xie et al., 2006, Pharm. Res. 23:1817-1826). In this method a thin stream of polymer solution is introduced at a constant flow rate into a strong electrostatic field. This field causes the movement of positive and negative ions present in the polymer solution. The charge of the solution is the difference in the numbers of positive and negative ions in a given region. This charge creates an electrical field within the polymer solution due to ion migration which leads to droplet instability when the electrical forces overcome the surface tension forces. When this happens a thin stream of charged polymer solution is projected from the Taylor cone and is collected on a grounded collection plate some distance from the site of polymer injection. As the polymer stream moves towards the collection plate the stream is stretched by repulsive forces causing the evaporation of the solvent, resulting in a mat of nano- to micro-diameter polymer fibers (Jia et al., 2002, Biotechnol. Prog. 18:1027-1032; Ge et al., 2004, J. Am. Chem. Soc. 126:15754-15761; Murugan et al., 2006, Tissue Eng. 12:435-447).

By varying process and material properties, fibers with different characteristics can be created. The variable process parameters include: applied voltage, distance from nozzle to collector, nozzle size, polymer feed rate, and apparatus setup. Material properties include: polymer composition, polymer concentration, solvent, conductivity, and viscosity (Yang et al., 2005, Biomaterials 26:2603-2610; Yoshimoto et al., 2003, Biomaterials 24:2077-2082; Kwon et al., 2005, Biomaterials 26:3929-3939). These mats have extremely high surface to volume ratios which can be further increased through the production of porous fibers making them ideal for many biological applications.

The present invention further contemplates a layer of the drug delivery device designed to become porous at a specific time post-implantation, for example, by including a degradable material (e.g., one of the biodegradable polymers above) into the pores of a slower degrading or biostable material. One specific example of such a layer is a polymer-ceramic hybrid material in which the polymer is biodegradable.

In accordance with an aspect of the invention, medical devices are contemplated in which a porous layer, such as those described above, among others, lies over a therapeutic-agent-containing region. Consequently, upon implantation or insertion of the device, therapeutic agent is allowed to diffuse from the underlying therapeutic-agent-containing region, through fluid (e.g., bodily fluid) within the pores of the porous layer, rather than having to diffuse though the solid material making up the porous layer.

Therapeutic agents”, “pharmaceuticals,” “pharmaceutically active agents”, “drugs” and other related terms may be used interchangeably herein and include genetic therapeutic agents and non-genetic therapeutic agents. Therapeutic agents may be used singly or in combination.

### B. Drug Loading

In accordance with the present invention, the drug agents are dissolved in a volatile organic solvent such as, for example, ethanol, isopropanol, chloroform, acetone, pentane, hexane, or methylene chloride, to produce a drug solution. In the case of Paclitaxel the preferred solvent is chloroform. The drug solution is then applied to the polymer. A volatile organic solvent typically is selected to provide drug solubilities much greater than the corresponding aqueous solubility for the substantially water-insoluble drug. Accordingly, application of the drug solution to the polymer often results in drug loadings that are orders of magnitude greater than loadings that can be achieved by application of a saturated aqueous solution of the drug to the polymer.

The drug solution. is applied to the polymer coating by any suitable means, including dipping the polymer coating into the drug solution or by applying the solution onto the coating such as by pipette or by spraying, for example. In the former method, the amount of drug loading is controlled by regulating the time the polymer is immersed in the drug solution, the extent of polymer cross-linking, the concentration of the drug in the solution and/or the amount of polymer coating. In another embodiment of the invention, the drug is incorporated directly into the polymer prior to the application of the polymer topcoat as a coating onto a medical device.

After applying the drug solution to the polymer coating, the volatile solvent is evaporated from the coating, for example, by drying in air or in an oven.

The present invention should be construed to encompass the use of compositions and methods of the present invention in combination with other systemically administered treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone), steroids (including prednisone and dexamethasone), and antidepressants (including gabapentin, amitriptyline, imipramine, doxepin) antihistamines, antitussives, muscle relaxants, brondhodilaters, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Drug-Loaded Microparticles

Microparticles fabrication was accomplished using techniques well-known in the art. Briefly, Paclitaxel was dissolved in a small amount of organic solvent. Drug solution was added to partially dissolved PLGA in organic solvent. Once PLGA was dissolved, the organic phase was brought up to volume. The external aqueous phase was stirred to form the drug loaded microparticle.

### Electrospinning

Traditional electrospinning techniques well known in the art were used to create a family of PLGA fibrous mats. Briefly, Paclitaxel and PLGA were dissolved in an organic solvent (e.g. DMF). The drug/polymer solution was transferred to a syringe and electrospun using an applied voltage and a grounded collection plate.

Different families of electrospun polymer fibers are created by changing the polymer MW, polymer concentration, solvent, and processing parameters. Some of the processing parameters that can be varied include: collection plate distance, applied electrical field, injection rate, and spinning time. The morphology of each family of electrospun polymer fibers was determined because some structural characteristics will have significant effects on the drug release rates. For example, a more porous fiber will probably result in faster diffusion times of both water into the polymer and drug out. Also, a smaller diameter may lead to faster bulk erosion of the fiber due to increased hydrolytic cleavage.

The fiber diameter and surface features were characterized using ESEM. The average fiber diameter was determined by measuring the diameter of a number of fibers and taking the arithmetic average.

The drug loaded polymer mat was then embedded in a permanent PVA macroporous hydrogel to create a composite device.

### Drug Loading

In order to determine drug loading, the fibrous mats were dissolved in a solvent such as dichloromethane and a common extraction procedure followed. This extraction procedure is similar to that used to determine drug loading of PLGA microparticles. A known amount of loaded particles was dissolved in an organic solvent to allow for the release of the encapsulated drug from the polymer matrix. A 50/50 (v/v) mixture of acetonitrile and H₂O was then added. After thorough mixing, the solution was subjected to a nitrogen purge until the dichloromethane (or other water insoluble solvent) evaporated. Since dichloromethane is not soluble in aqueous solutions, it will form a cloudy mixture and upon evaporation the solution will become clear. The freed drug and polymer are both be present in the acetonitrile/H₂O phase upon evaporation of the organic phase. High-performance liquid chromatography (HPLC) was performed on a sample of the acetonitrile/H₂O solution and the drug concentration determined. With knowledge of the solution volume, the initial amount of loaded material, and the drug concentration, the total drug loading can be determined.

### PVA Hydrogels

Polyvinyl alcohol hydrogels were fabricated using a solvent-free technique that utilized freeze-thaw cycles to achieve crosslinking. Aqueous solutions of PVA (MW 113,000) were made and then poured into cylindrical molds which were then subsequently frozen and thawed. Previous work in our laboratory has shown satisfactory crosslinking with 6 days of freeze-thaw cycles. The number of cycles can be decreased to achieve less crosslinking. The affect of freeze-thaw cycles and thus crosslinking amount were determined using mechanical stability tests completed with an Instron. Key distance between crosslinks was determined from mechanical stability data.

The porosity of the virgin PVA hydrogels was determined using both experimental and theoretical techniques. Mercury intrusion porosimetry is a common experimental technique for determining porosity. There are also theoretical techniques available for estimating material porosity according to various other parameters.

Another important characteristic that will affect drug release kinetics is the interconnectivity of the porous network within the hydrogel. Interconnectivity was determined using a very simple method utilizing carbon dye. Carbon black was added to hydrogels and then centrifuged. The gels were removed and cut in order to facilitate examination using ESEM. Upon examination the ratio of pores which are colored to those which are not was determined. Black coloring indicates pore connectivity to the surface of the hydrogel, either directly or indirectly though channel interconnectivity. A lack of black coloring indicates that the pore was internal only and had no channel connectivity.

### Fibrous Mats in PVA Hydrogels

The affect on surface morphology was determined through visualization using ESEM. Fiber distribution within the PVA hydrogels was determined using fluorescently marked coumarin-6 loaded PLGA fibers and confocal microscopy techniques. Drug loading was determined indirectly using the fiber drug loading and the hydrogel fiber loading. Any fibers not successfully incorporated into the hydrogels were subtracted from the total amount initially loaded.

The polymer interaction between the PLGA fibers and the PVA hydrogel was determined by completing degradation studies. Fluorescent-marked fiber loaded PVA hydrogels as well as empty PVA hydrogels were immersed in PBS baths at 37° C. for various lengths of time. At specific time points samples were removed and their properties tested. The mass of the samples at various times were recorded and mechanical tests completed to elicit structural properties such as distance between crosslinks. The surface morphology and fiber distribution of the samples was visualized using ESEM and confocal respectively. Porosity was determined using theoretical and experimental techniques, and pore interconnectivity determined using carbon black experiments followed by ESEM visualization.

### Drug Release Kinetics

Drug release was characterized by withdrawing small samples (0.3 ml) and replacing fresh PBS. The total amount released was calculated using the following equation (assuming 20 ml release medium):

$${M}_{tn} = 20 ml ( {C}_{n} \frac{µ g}{ml} ) + 0.3 ml ( {\sum }_{n = 1}^{n - 1} {C}_{n} )$$

Due to the extremely poor solubility of Paclitaxel in aqueous solutions, the release medium also contained 1% Tween 80, an emulsifier which increases solubility. The release medium was extracted, then run in HPLC to determine concentration. Extraction standards are done and a standard Paclitaxel curve generated.

The results of the experiments presented in this Example are now described.

### Example 1

### Drug Release Profiles for Polymers Formed by Electrospinning

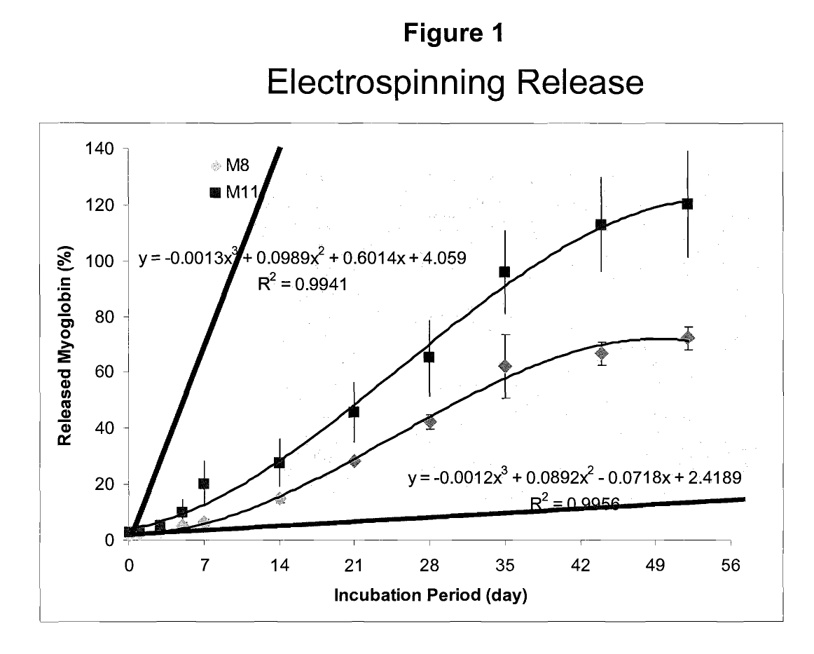

The drug delivery device contemplated in the present invention encompasses the full range of all release profiles depicted in FIG. 1 where between 0 and 100% of the drug of interest is release between day 0 and day 100 post-implantation.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A sustained release local delivery system comprising a vertebral replacement cage and a drug delivery component, wherein said drug delivery component comprises a hydrogel exo-structure having at least one biocompatible polymer.

2. The local delivery system of claim 1, wherein said hydrogel is nondegradeable.

3. The local delivery system of claim 1, wherein said biocompatible polymer comprises a fibrous mat.

4. The local delivery system of claim 3, wherein said mat is made from an electrospinning process.

5. The local delivery system of claim 3, wherein said polymer comprises polyvinyl alcohol, poly(lactide), poly(lactide-co-glycolide), or a combination thereof.

6. The local delivery system of claim 3, wherein each said mat comprises at least one pharmaceutical agent.

7. The local delivery system of claim 6, wherein said pharmaceutical agent comprises Paclitaxel.

8. The local delivery system of claim 6, wherein said pharmaceutical agent is released at a rate of at least 8.5% per day.
